(19) United States
(12) Patent Application Publication
(10) Pub. No.: US 2023/0414501 A1
(43) Pub. Date: Dec. 28, 2023

(54) BROAD SPECTRUM CANNABINOID AND TERPENE MINERAL SALVE, METHOD OF USING, AND METHOD OF MAKING THE SAME

(71) Applicant: Holistic Hemp Solutions, Inc., Norton, MA (US)

(72) Inventor: Julie A. Mejia, Norton, MA (US)

(21) Appl. No.: 18/331,443

(22) Filed: Jun. 8, 2023

Related U.S. Application Data
(60) Provisional application No. 63/367,131, filed on Jun. 28, 2022.

(51) Int. Cl. A61K 9/06; A61K 31/00; A61K 33/14; A61K 47/44

(52) U.S. Cl. CPC A61K 9/06; A61K 31/658; A61K 33/14; A61K 47/44

(57) ABSTRACT

A CBD mineral salve containing hemp oil, at least one carrier oil, and suspended magnesium is disclosed. Methods of using and making the CBD mineral oil are also disclosed.

### CROSS-REFERENCE TO RELATED APPLICATIONS

This application claims priority to U.S. Provisional Application No. 63/367,131, filed on Jun. 28, 2022. The entire contents of the foregoing application is incorporated by reference herein.

### FIELD OF THE DISCLOSURE

The disclosure relates to a mineral salve containing cannabinoids and terpenes, and a carrier oil prepared using a heated, blending process to achieve the desired characteristics, and which may be used to prevent or treat arthritis, muscle aches, inflammation, pains, and/or skin conditions.

### BACKGROUND OF THE DISCLOSURE

Cooling remedies for aches and pains have been used for thousands of years. For instance, mint oil, which contains the cooling agent menthol, has been used in traditional healing practices. In present day, people most commonly use products like BenGay® and IcyHot® to ease muscle pain, minor arthritis, and to cool irritation and inflammation. Active ingredients menthol and methyl salicylate are counterirritants that work by causing the skin to feel cool and then warm. These feelings on the skin distract the user from feeling the aches/pains in the muscles, joints, and/or tendons.

Most sports creams contain an active ingredient methyl salicylate, or salicylic acid, a toxic chemical which, if overused, can result in serious injury or, even in rare cases, in death from poisoning. As an anti-clotting agent, at high doses, salicyclic acid can cause internal bleeding, arrhythmias of the heart, and/or problems in the liver. “Bengay Death Highlights OTC Dangers,” ABCNews, Mar. 24, 2008 (https://abcnews.go.com/Health/Healthday/story?id=4507580&page=1).

In addition, these common topical creams are more effective for acute pain resulting directly from tissue damage, such as a torn or pulled muscle. Treatment of neuropathic, or nerve-related, pain, is much more difficult because the injured nerves seem to generate pain signals without an external influence. Neuropathic pain originates, in part, from an intricate mesh of sensory neurons, which is not thoroughly understood. Chu, J., “The Mystery of BenGay: A study reveals how age-old cooling remedies help to alleviate nerve-related pain.” MIT Tech. Rev., Aug. 22, 2006 (https://www.technologyreview.com/2006/08/22/273714/the-mystery-of-bengay/#:—:text=Products %2Olike %20BenGay %20 are %20modern,a %20burn %2000/020pulled % 20muscle).

There is a need for a safer and natural pain-relieving topical cream that relieves both acute path and neuropathic pain.

### SUMMARY OF THE DISCLOSURE

A mineral salve is disclosed containing: a) hemp oil containing cannabinoids and optionally terpenes; b) magnesium oil; c) at least one carrier oil; d) turmeric oil or curcumin oil; e) black pepper oil; f) a natural preservative; and g) one or more essential oils. The magnesium oil is substantially uniformly suspended within the carrier oil. The magnesium oil may be present in about 5% to about 40% by volume of the CBD mineral salve. The hemp oil may be present in about 5% to about 20% by volume of the CBD mineral salve. The carrier oil may be present in about 60% to about 80% by volume of the CBD mineral salve, and may be selected from: bee wax, carauba wax, Olive oil, Hempseed oil, Coconut oil, Avocado oil, Coco Butter, Mango Butter, Shea Butter (raw yellow, refined white), Jojoba, and any combination thereof. The carrier oil may comprise bee wax or carauba wax, and at least one additional carrier oil selected from the group consisting of: Olive oil, Hempseed oil, Coconut oil, Avocado oil, Coco Butter, Mango Butter, Shea Butter (raw yellow, refined white), Jojoba, and any combination thereof. The one or more essential oil is present in about 2% to about 10% by volume of the CBD mineral salve, and may be selected from the group consisting of: eucalyptus, peppermint, citrus, lemongrass, lavender, and any combination thereof. The CBD mineral salve may include a natural preservative, which may be selected from the group consisting of: Populus tremuloides bark extract; willow bark extract; leuconostoc ferment filtrate; Phytocide Elderberry; a combination of gluconolactone and sodium benzoate; a combination of benzyl alcohol, salicylic acid, glycerin, and sorbic acid; radish root; lactobacillus ferment; and any combination thereof.

Methods of using the CBD mineral salve are disclosed for reducing or treating arthritic pain or muscle pain, inflammation, and/or a skin condition by topically administering the CBD mineral salve, optionally one to six times per day. The skin condition may be an inflammatory skin condition.

A method of making the CBD mineral salve is disclosed which includes the steps of: supplying raw hemp; processing the raw hemp to make hemp oil; supplying magnesium chloride flakes; processing the magnesium chloride flakes with water to make magnesium oil; melting the at least one carrier oil to a liquid state; mixing the hemp oil, the melted carrier oil, black pepper oil, turmeric oil or curcumin oil, the natural preservative, and the one or more essential oils in a blending unit; and blending the mixture for an amount of time at about 120° C. to about 170° C. to provide a substantially uniformly blended CBD mineral salve. The magnesium oil may be substantially uniformly suspended in the carrier oil.

### DETAILED DESCRIPTION OF THE DISCLOSURE

The CBD mineral salve disclosed herein includes magnesium oil, turmeric oil and/or curcumin oil, black pepper oil, one or more carrier oils, one or more essential oils, and hemp oil, which includes cannabinoids and terpenes. The CBD mineral salve may include magnesium oil made from water and magnesium chloride flakes, organic turmeric oil, organic black pepper oil, a natural preservative (e.g., lactobacillus ferment), and at least one essential oil for fragrance. Mineral salve, as used herein means an ointment used to soothe the surface of the body, containing the mineral, magnesium, a unique mineral to include in any topical.

With this novel CBD mineral salve multiple active compounds are combined in a synergistic and unique manner to target and reduce pain and inflammation and have antibacterial and antimicrobial properties in one product. Because each individual subject reacts differently to different active compounds, by containing multiple ingredients that target pain and inflammation, at least one or more of these ingredients will be effective with the subject's specific biological makeup to target pain, inflammation and healing. For example, some subjects may experience relief from the magnesium but not process the CBD, while others experience the opposite; at least one or more of the ingredients will assist and work with the subject's body.

Further, the inventors developed a unique method of suspending the magnesium oil, which is water-based, in an oil-based system to provide a uniformly potent and smooth product. This is an advancement over other products in the market and from a chemistry perspective.

The CBD mineral salve may be applied safely to pediatric and adolescent patients, adults of all ages, and animals that may need the benefits this product can provide. It offers a natural, holistic means to reduce pain, inflammation and skin conditions.

Magnesium oil is made from a mixture of magnesium chloride flakes, or magnesium chloride salt, and water. When these two substances are combined, the resulting liquid has an oily feel, but isn't technically an oil. The magnesium oil may be made from magnesium chloride flakes or salts. Magnesium oil may be purchased directly from a supplier, or made from processing magnesium chloride flakes or salts and processed as disclosed herein. For example magnesium chloride flakes may be purchased from a supplier, such as those sold by Jedwards International, Inc. a wholesale bulk supplier of Natural Magnesium Chloride—Dead Sea (https://bulknaturaloils.com/natural-magnesium-chloride-dead-sea.html). The magnesium oil may be made from magnesium chloride flakes and water. Research has shown that topical application of magnesium has better bioavailability and breakdown/absorption when it is processed from magnesium chloride flakes rather than from salt.

The CBD mineral salve may include less than about 40% by volume of magnesium oil. The CBD mineral salve may include less than about 40% by volume of magnesium oil. The CBD mineral salve may include about 5% to about 40%, about 5% to about 20%, about 30% to about 40%, about 5% to about 15%, or about 10% by volume of magnesium oil. The amount of magnesium oil in the CBD mineral salve may be suitable for use up to 6 times daily. The amount of magnesium oil should not affect the subject's daily intake or exceed recommended dosages of magnesium.

In the inventive CBD mineral salve, the magnesium oil is substantially uniformly suspended within the oil blends therein. The magnesium does not settle out over time, for example over a period of about 1 year or about 2 years. The particles of the magnesium oil may have a mean particle size of about 500 μm to about 800 μm. This uniform suspension of the magnesium oil, which is water-based, is extremely challenging and difficult to attain. The synergy of the substantially uniformly suspended magnesium oil with all the other anti-inflammatory components of the CBD mineral salve creates a product that directly targets pain and inflammation in a subject. Substantially uniformly suspended may mean that the heated blending element breaks down the magnesium oil on a molecular level with the other oil-based compounds and, as it cools, the particles of magnesium oil are suspended within the structure of the CBD mineral salve substantially evenly.

To create the desired texture and suspension, the magnesium oil, which is water-based, may be made from magnesium chloride flakes. Water, preferably hot or at or near boiling, and magnesium chloride flakes may be mixed in about a 2:1 to about a 1:2 ratio, or in about a 1:1 ratio, to dissolve the magnesium chloride flakes. The mixture may be agitated by any means until the flakes are dissolved to provide the magnesium oil. The magnesium oil may be allowed to rest and come to room temperature before further processing into the salve.

Any turmeric oil or curcumin oil may be used in accordance with this disclosure. The turmeric oil or curcumin oil may be organic. The CBD mineral salve may include a steam distilled organic turmeric oil that has 100% purity. Curcumin, a yellow coloring agent extracted from turmeric, is known to have strong anti-oxidative and anti-inflammatory activities when used as a remedy for the prevention and treatment of chronic diseases. The CBD mineral salve may include less than about 3% by volume of turmeric oil or curcumin oil. The CBD mineral salve may include about 0.5% to about 3% by volume, about 1% to about 3% by volume, about 1.5% to about 2.5% by volume, or about 2% by volume of turmeric oil or curcumin oil.

Any black pepper oil may be used in accordance with this disclosure. The black pepper oil may be organic. The mineral salve may include a steam distilled organic black pepper oil that has 100% purity. Black pepper oil possesses anti-inflammatory and anti-spasmodic properties. It can assist with reducing pain from a number of ailments. It is known for assisting with easing cramps, improving tendonitis/bursitis, as well as symptoms of arthritis and rheumatism. The warming effect can improve blood circulation when applied transdermally. Black pepper oil is antibacterial, antifungal and antimicrobial, which helps assist with a variety of bacterial and fungal skin infections and/or conditions. It is a compound that contains high amounts of antioxidants, including piperine, which may help prevent free radical damage to cells. Other benefits include assistance with blood sugar stabilization, brain functionality improvements, improving cholesterol levels and even has anti-cancer properties. Research has also shown black pepper oil supports the absorption of turmeric/curcumin. As black pepper supports diabetes, it specifically helps tackle neuropathic pain caused by diabetes when applied topically. It also can serve as cleansing tool, riding the body of built up acid within the muscle tissue. Black pepper has also been known to assist with asthma and serve as an expectorant. The CBD mineral salve may include less than about 3% by volume of black pepper oil. The CBD mineral salve may include about 0.5% to about 3% by volume, about 0.5% to about 2% by volume, about 0.5% to about 1.0% by volume, or about 1% by volume of black pepper oil.

The hemp oil contains cannabinoids. The hemp oil may contain cannabinoids and terpenes. The hemp oil may be concentrated. One means to obtain concentrated hemp oil is by using a heat source applied to the raw hemp material placed in a closed loop extraction system with a CO₂ component used as a solvent for extraction resulting in higher amounts of CBD in the resulting help oil. The hemp oil may be broad spectrum. Broad spectrum means that there are multiple terpenes and multiple cannabinoids present in the hemp oil. However, when a heat source is used for extraction, some terpenes and/or cannabinoids are lost due to the decarboxylation process.

The hemp oil may include one or more of the following: Myrcene, Beta-caryophyllene, Limonene, Linalool, Pinene, Humulene, Terpinolene, Alpha-bisabolol, Eucalyptol, Geraniol, Terpineol, Farnesene, Borneol, Ocimene, Nerolidol, Guaiol, Valencene, Delta-3 carene, Phytol, Sabinene, Phellandrene, Menthol, Fenchol, Terpinene, Isoborneol, Cymene, Octanol, Cedrene, Camphene, Geranyl Acetate, Bergamotene, Camphor, Pulegon, CBD, CBDa, CBDva, CBG, CBGa, THCa, THCv, CBGv, CBGva, CBDv, CBCv, CBC, CBCa, CBGM, CBN, CBL, CBLa, and any combination thereof.

The hemp oil may contain about 60 wt % to about 97 wt % total cannabinoids, about 60 wt % to about 90 wt % total cannabinoids, about 65 wt % to about 95 wt % total cannabinoids, about 70 wt % to about 90 wt % total cannabinoids, or about 75 wt % to about 86 wt % total cannabinoids.

The hemp oil may contain one or more of the following cannabinoids: Δ9-THC, THCv, CBD, CBDv, CBC, CBG, CBGa, CBN, THCa, Δ8-THC, THCva, CBL, CBLa, CBCv, and CBT. The hemp oil may contain cannabinoids selected from the group consisting of: CBD, CBDv, CBC, CBG, CBN, and any combination thereof. The hemp oil may include CBD, CBDv, CBC, CBG, and CBN. The hemp oil may include CBD, CBDv, CBG, and CBN, and other cannabinoids in less than about 0.1 wt % each, The hemp oil may contain less than about 0.1 wt % of one or more of the following cannabinoids: Δ9-THC, CBNa, THCv, THCva, CBCv, CBT, CBCa, CBL, CBLa, THCa, CBDa, CBDva, CBGa, and Δ8-THC. The hemp oil may contain less than about 0.03 wt % of one or more of the following cannabinoids: Δ9-THC, THCv, THCa, CBDa, CBGa, and Δ8-THC. The hemp oil may contain less than 0.3 wt. % of Δ8-THC and Δ9-THC. The hemp oil may contain less than 0.3 wt. % of Δ9-THC.

The hemp oil may contain about 60 wt % to about 95 wt %, about 60 wt % to about 93 wt %, about 80 wt % to about 95 wt %, about 60 wt % to about 85 wt %, about 65 wt % to about 80 wt %, or about 70 wt % to about 80 wt % CBD. The hemp oil may contain about contain about 0.3 wt % to about 2.0 wt %, about 0.4 wt % to about 1.5 wt %, about 0.4 wt % to about 1.0 wt %, or about 0.5 wt % to about 1.2 wt % CBDv. The hemp oil may contain about contain about 0.3 wt % to about 2.5 wt %, about 0.7 wt % to about 2.0 wt %, or about 1.0 wt % to about 2.0 wt % CBG. The hemp oil may contain about 0.3 wt % to about 2.5 wt %, about 0.5 wt % to about 2.0 wt %, about 0.5 wt % to about 1.0 wt %, about 0.7 wt % to about 2.0 wt %, or about 0.9 wt % to about 1.5 wt % CBN. The hemp oil may contain about 80 wt % to about 95 wt % CBD, about 0.3 wt % to about 2.0 wt % CBDv, about 0.7 wt % to about 2.0 wt % CBG, and about 0.3 wt % to about 2.0 wt % CBN. When present, the hemp oil may contain about 1.0 wt % to about 4.5 wt %, about 1.5 wt % to about 3.8 wt %, or about 2.5 wt % to about 3.5 wt % CBC.

The CBD mineral salve may include about 5% to about 20%, about 5% to about 15%, or about 10% by volume of hemp oil. The hemp oil may be made by heat extraction processing of raw hemp. A CBD mineral salve was made by processing one or more strains of raw hemp using heat extraction to make a heat extracted concentrated hemp oil. The amount of different strains used to make batches varies based on desired effect. The hemp may be selected from the following strains: Hawaiian Haze, Sour Space Candy, Super Sour Space Candy, Cherry Pie, Special Sauce, Lifter Plus, Elektra, Cherry Creme Brulee, Harlequin, Gorilla Glue (Hemp version), Hulk, Chardonnay, Cherry Wine, Bubba Kush (Hemp version), Haute Wife, Cheese (Hemp version), Santa Clara Haze, Gun Powder OG (Hemp version), Silver Haze (CBG Hemp version), Cherry #4, T-1 (Hemp version), White (CBG Hemp version), Sour G (CBG Hemp version), White Widow (CBG Hemp version), Jack Frost (Hemp version), Dessert Snow (CBG Hemp version), Lemon Octane, Blue Dream (Hemp version), Spec Diesel, Blueberry Muffin, Strawberry cake, Cherry Abcus, Magic Bullet, Pink Panther, Maui Wowi, Golden Kush, Red Bordeaux, Frosted Lime, Boaox, Bubblegum (Hemp version), Merlot, Super Lemon Haze (Hemp version), John Snow (Hemp version), Superglue (Hemp version), Lemon Cream Diesel, Stem Cell, White Whale, Frosted Cake, Stardust, Plain Jane Secrets Nature, Shiatsu, Matterhorn, Forbidden Fruit, Forbidden V, Zkittles (Hemp version), Bubba Cryo, Runtz (Hemp version), Pine Walker, and any combination thereof. The one or more selected strains of hemp may contain less than 0.3% Δ9-THC.

The raw hemp may be processed by extraction by any means known in the art to make a hemp oil. The raw hemp may be processed by extraction with organic ethanol, food grade ethanoyl, or CO₂. When using ethanol for extraction, the raw hemp material is processed through a closed loop system, heat is applied and the evaporation process begins; at the end of this processing, heat is applied again and the majority of liquid components are evaporated and/or separated. The hemp oil may have a shelf life of over 2 years so it may be prepared and stored before incorporation into the CBD mineral salve. The hemp oil may be bottled and stored in any sized cobalt blue jar which reduces degradation of the product by shielding it from light.

The carrier oil may be selected based on user preference and which oil delivers the best effect/metabolizes best for the subject's endocannabic system and body. The carrier oil may be all natural or naturally derived oil, all natural or naturally derived butter, all natural or naturally derived wax, or any combination thereof. The carrier oil may be selected from the group consisting of: Olive oil, Hempseed oil, Fractionated MCT/Coconut oil, Avocado oil, Bee and Carnauba Waxes, Coco Butter, Mango Butter, Shea Butter (raw yellow, refined white), Shea oil, Jojoba, Cod Liver oil, Sunflower oil, Peanut oil, any known vegetable oil, such as but not limited to canola or safflower, Almond butter, Almond oil, Glycerin (in all forms), Liquid Castile Soap, Emu and other animal derived oils, Vitamin E oil, Caster Oil, Zinc Oxide, Citric Acid, and any combination thereof. The carrier oil may be selected from the group consisting of: Olive oil, Hempseed oil, Fractionated MCT/Coconut oil, Avocado oil, Almond oil, Cod Liver oil, Sunflower oil, Peanut oil, shea butter, any known vegetable oil, such as but not limited to canola or safflower, or any combination thereof. The carrier oil may be an olive oil blend containing olive oil and one or more additional carriers. The carrier oil may be olive oil, shea butter, coconut oil, Bee and Carnauba Waxes, and any combination thereof. The carrier oil may include bee or carauba wax, and at least one of: Olive oil, Hempseed oil, Coconut oil, Avocado oil, Coco Butter, Mango Butter, Shea Butter (raw yellow, refined white), Jojoba, and any combination thereof. Carnauba wax may improve stability and texture (e.g., softness) of the product. Carnauba wax is vegan and made from sustainable trees and do not disturb the bee population/and or their habitat.

The CBD mineral salve may contain about 60% to about 80%, about 65% to about 75%, or about 68% to about 72% by volume of a carrier oil. The CBD mineral salve may contain about 2% to about 10%, or about 5% by volume of carnauba wax, optionally in combination with another amount of an additional carrier oil. Carnauba wax may be added as a natural stabilizer, and because of its properties as a soft, vegan, and sustainable product.

The CBD mineral salve may contain one or more natural additives. The additive may be 100% natural. The natural additive may be a natural or naturally derived coloring agent, fragrance or preservative. The additive may be natural or naturally derived mint or spearmint. When present, the CBD mineral salve may contain about 0.5 wt. % to about 20 wt. %, about 1 wt. % to about 10 wt. %, about 11 wt. % to about 5 wt. % of one or more additives. The CBD mineral salve may include a natural preservative. The natural preservative may include Populus tremuloides bark extract (also referred to as Phytocide Aspen Bark Powder), willow bark extract, leuconostoc ferment filtrate (e.g., Arborcide® OC), Phytocide Elderberry (a natural preservative derived from the fruit of the Elderberry tree), a combination of gluconolactone and sodium benzoate (e.g., NeoDefend™, Geogard Ultra®), a combination of benzyl alcohol, salicylic acid, glycerin, and sorbic acid (e.g., Preservative ECO, Geogard® ECT), radish root, lactobacillus ferment, and any mixture thereof. Natural preservatives including radish root and/or lactobacillus ferment may include, but are not limited to, Leucidal® Liquid SF, Leucidal® SF Complete, Leucidal® Liquid Complete, Leucidal® SF Max, and AMTicide® Coconut. The CBD mineral salve may contain about 0.3 wt. % to about 2 wt. %, about 0.5 wt. % to about 1 wt. % of a preservative. The CBD mineral salve may be free of artificial additives, including but not limited to artificial flavors, fragrances, colors, preservatives, and thickeners.

The CBD mineral salve may contain about 0.4 wt % to about 2.0 wt %, about 0.4 wt % to about 1.5 wt % total cannabinoids, about 0.5 wt % to about 1.2 wt % total cannabinoids, about 0.7 wt % to about 1.0 wt % total cannabinoids, or about 0.9 wt % to about 1.0 wt % total cannabinoids. The CBD mineral salve may contain about 0.1 wt % to about 1.2 wt %, about 0.7 wt % to about 1.0 wt % CBD, or about 0.9 wt % to about 1.0 wt % CBD. The CBD mineral salve may contain about wt % to about 0.1 wt %, about 0.01 wt % to about 0.5 wt %, or about 0.02 wt % to about 0.04 wt % CBC. One ounce of the CBD mineral salve may contain about 200 mg to about 300 mg, or about 250 mg of total cannabinoids.

The CBD mineral salve may contain less than about 0.1 wt % of one or more of the following cannabinoids: Δ9-THC, CBN, CBNa, THCa, Δ8-THC, THCv, THCva, CBDv, CBL, CBLa, CBCv, and CBT. The CBD mineral salve may contain less than about 0.1 wt % of all of the following cannabinoids: Δ9-THC, CBN, CBNa, THCa, Δ8-THC, THCv, THCva, CBDv, CBL, CBLa, CBCv, and CBT. The CBD mineral salve may contain less than about 0.01 wt % of one or more of the following cannabinoids: Δ9-THC, CBN, CBNa, THCa, Δ8-THC, THCv, THCva, CBDv, CBL, CBLa, CBCv, and CBT. The CBD mineral salve may contain less than about 0.01 wt % of all of the following cannabinoids: Δ9-THC, CBN, CBNa, THCa, Δ8-THC, THCv, THCva, CBDv, CBL, CBLa, CBCv, and CBT. The CBD mineral salve may contain less than 0.3 wt. % of Δ8-THC and Δ9-THC. The CBD mineral salve may contain less than 0.3 wt. % of Δ9-THC.

The essential oil may be any known for use in the art. The essential oil may be added for fragrance. The essential oil may be any natural fragrance oil, such as but not limited to, eucalyptus, peppermint, citrus (e.g., tangerine, orange, lemon, grapefruit, lime), lemongrass, lavender, and any combination thereof. The CBD mineral salve may include about 0.5% to about 5%, or about 1% to about 2% by volume of essential oil.

The CBD mineral salve may be topically applied to a subject, e.g., human or animal, at room temperature. The CBD mineral salve may be topically applied to a subject to reduce or treat pain (e.g., arthritic pain or muscle pain), inflammation, wound healing, and/or skin conditions. The dosing regimen may be about 1 ml to about 3 ml, or enough to cover the area of the skin or body at issue, applied topically to the infected, or irritated skin, or area of pain or inflammation, one to six times daily, or as needed. The dosing regimen may change over time. For example, the user may begin with applying the CBD mineral salve topically once per day and increase the frequency to six times per day over some period of time.

A method is disclosed for treating skin conditions of a subject by applying the CBD mineral salve disclosed herein to the skin of the subject. The skin condition may be an inflammatory skin condition, such as granuloma annulare, eczema, seborrheic dermatitis, and psoriasis. The skin condition may be a response to gluten on a subject having celiac's disease.

A method of reducing or treating arthritic pain or muscle pain, or inflammation in a subject may comprise applying the CBD mineral salve topically to the area of pain or inflammation as needed, or about every six to two hours until pain subsides. A method of reducing or treating arthritic pain or muscle pain, or inflammation may comprise applying the CBD mineral salve topically to the area of pain or inflammation one to six times per day for a period of days, in combination with administration of a tincture disclosed in co-owned U.S. Provisional Application No. 63/317,717, filed on Mar. 8, 2023, the entire contents of which are incorporated herein.

A method of reducing or treating neuropathic discomfort that may be caused by diabetes and/or pain disorders such as: RSD otherwise known as Reflex Sympathetic Dystrophy and/or Complex Regional Pain Syndrome, in a subject may comprise topically applying the CBD mineral salve to the area of discomfort. The methods of administration and dosing may be the same as described above.

A method of disinfecting and/or healing a wound, cut, or skin infection in a subject may comprise topically applying the CBD mineral salve to the wound, cut, site of cannula removal, or skin infection. The methods of administration and dosing may be the same as described above. Applying the CBD mineral save may improve post-surgical success at the stitching/incision site and help wound healing while reducing pain and swelling after surgery. As such, the CBD mineral salve may be a substitute for vaseline/petroleum based products as some subjects do not process those products well as they are created from crude oil and hydrocarbons contained in natural gas.

A method of treating and/or healing a burn on skin of a subject may comprise topically applying the CBD mineral salve to the burn. The burn may be a first degree or second degree burn. The methods of administration and dosing may be the same as described above.

Other methods include administering the CBD mineral salve to treat or reduce anxiety, or inflammation. The methods of administration may be the same as described above.

A method of making a CBD mineral salve is disclosed. That method includes: i) supplying raw hemp; ii) processing the raw hemp to make concentrated hemp oil; iii) supplying magnesium chloride flakes; iv) processing the magnesium chloride flakes with water to make magnesium oil; v) melting at least one carrier oil to a liquid state; vi) mixing the concentrated hemp oil, the melted at least one carrier oil, black pepper oil, turmeric oil or curcumin oil, optionally, also a natural preservative, and one or more essential oils in a blending unit; v) blending the mixture for an amount of time at about 120° C. to about 170° C. to provide a substantially uniformly blended CBD mineral salve. The step of processing of raw hemp to make hemp oil is the same as set forth above. The step of processing of magnesium chloride flakes to make magnesium oil is the same as set forth above. The step of blending the mixture may performed for about 5 to about 20 minutes, or about 10 minutes. Blending the mixture may be performed for at a medium heat temperature of about 150° F. to obtain a smooth and well-blended mixture.

In melting at least one carrier oil to a liquid state, one or more carrier oils may be selected at the same amount or in different amounts and then melted by application of heat. When multiple carrier oils are used, they may be melted sequentially one at a time before adding the next carrier oil or they may be mixed and then melted concurrently. Once all of the carrier oils are melted, the melted carrier oils may be transferred to a heated mixing/blending unit. The mixing/blending unit may be any such equipment known for use in the art to concurrently mix and heat, such as, but not limited to, a jacketed mixing vessel (https://www.adinathmachines.com/mixer-blender-and-stirrers/jacketed-mixing-vessel/), a Silvers on mixer (https://www.silverson.com/us/re source-library/application-rep orts/production-of-pharm aceutical-cream-and-ointments), or any commercial blender that also has a heating capability.

Shea butter and coconut oil may be selected as the carrier oils, added together and then melted on a double boiler until melted and smooth in texture. This may be followed by the addition of bee wax or carnauba wax, which is then melted into the mix of melted shea butter and coconut oil. Once all the carrier oils are melted down, they may be transferred to a heated mixing/blending unit.

Next, hemp oil, the melted at least one carrier oil, black pepper oil, turmeric oil or curcumin oil, and one or more essential oil may be measured out, and added to the mixing/blending unit. Optionally, also an amount of a natural preservative, or other natural additive may be added to the mixing/blending unit. The ingredients may then be blended together for an amount of time, such as about 2 to about 10 minutes, at about 120° C. to about 170° C., about 130° C. to about 160° C., about 140° C. to about 155° C., or at about 155° C. to provide a substantially uniformly blended CBD mineral salve.

The CBD mineral salve may be measured into weighted or portions by volume and immediately placed into jars, for example, but not limited to, glass jars, and left to cool.

The features and advantages of the present disclosure are more fully shown by the following examples which are provided for purposes of illustration, and are not to be construed as limiting the invention in any way.

### EXAMPLES

### Example 1

A CBD mineral salve was made by first processing a mixture of strains of raw hemp using heat extraction to make a heat extracted concentrated hemp oil.

Next, a water-based magnesium oil was made from magnesium chloride flakes. Water and magnesium chloride flakes were combined in a one to one ratio. A cup of magnesium flakes was added to one cup of boiling water and stirred until the flakes dissolved. Once that was complete, it was set aside until its addition at the very end of the process.

Equal parts (80 grams) of both shea butter and coconut oil were melted down to a smooth texture. In small batches, this can be done on top of a double boiler system; on a larger scale, it can be procured in a kettle and/or a heated blending system. The carrier oil blend of shea butter and coconut oil was about 70 wt. % of the final mineral salve product.

After these ingredients are melted down, carnauba wax was added. The wax constituted about 5% of the total mineral salve composition. Once the carrier oils were melted, they were placed in a heated mixing/blending unit.

Next, 1.5 g hemp oil, 4 grams of essential oils, 1 gram of black pepper oil, 2.5 g of turmeric oil, 60 grams of magnesium oil, and 2 grams of lactobacillus ferment were added to the heated mixing/blending unit. All ingredients were blended together at a medium heat temperature of about 150° F. for about 10 minutes until the mixture was smooth and well blended. The blended mixture was immediately placed into glass jars and left to cool.

The CBD mineral salve prepared according to the process described above was 0.97 wt. % total cannabinoids. The cannabinoid content in shown in Table 1.

TABLE 1

| | mg per 56.7 g serving | percent by mass |
|---|---|---|
| Total Cannabinoids | 549.99 mg | 0.97% |
| CBD | 532.98 mg | 0.94% |
| CBC | 17.01 mg | 0.03% |

The following cannabinoids were not detected (i.e., less than 0.01%): Δ9-THC, CBN, CBNa, THCa, CBDa, CBDva, Δ8-THC, CBG, CBGa, THCv, THCva, CBDv, CBCa, CBL, CBLa, CB Cv, CBT.

### Example 2

A hemp oil was made by processing a mixture of strains of raw hemp using heat extraction to make a heat extracted concentrated hemp oil. The cannabinoid profile of the heat extracted concentrated hemp oil was tested and the results are shown in Table 2 below.

TABLE 2
Cannabinoid Profile and Potency

| ID | Wt. % | Conc. (mg/g) |
|---|---|---|
| Δ9-THC | ND | ND |
| THCV | ND | ND |
| CBD | 78.3 | 783 |
| CBDV | 1.06 | 10.6 |
| CBG | 1.89 | 18.9 |
| CBC | 3.36 | 33.6 |
| CBN | 1.22 | 12.2 |
| THCA | ND | ND |
| CBDA | ND | ND |
| CBGA | ND | ND |
| Δ8-THC | ND | ND |
| Exo-THC | ND | ND |
| Total | 85.8 | 858 |

### Example 3

Another hemp oil was made by processing a mixture of strains of raw hemp using heat extraction to make a heat extracted concentrated hemp oil. The cannabinoid profile of this heat extracted concentrated hemp oil was tested and the results are shown in Table 3 below. The following cannabinoids were not detected: Δ9-THC, CBNa, THCa, CBDa, CBDva, Δ8-THC, CBGa, THCv, THCva, CBC, CBCa, CBL, CBLa, CBCv, and CBT.

TABLE 3
Cannabinoid Profile and Potency

| ID | Wt. % |
|---|---|
| CBD | 93.00 |
| CBDV | 0.46 |
| CBG | 2.09 |
| CBN | 0.50 |
| Total* | 96.05 |

*sum of all detected cannabinoids

While there have been described what are presently believed to be various aspects and certain desirable embodiments of the disclosure, those skilled in the art will recognize that changes and modifications may be made thereto without departing from the spirit of the disclosure, and it is intended to include all such changes and modifications as fall within the true scope of the disclosure.

What is claimed:

1. A CBD mineral salve comprising:
a) hemp oil containing cannabinoids and terpenes;
b) magnesium oil;
c) at least one carrier oil;
d) turmeric oil or curcumin oil;
e) black pepper oil;
f) a natural preservative; and
g) one or more essential oils;
wherein the magnesium oil is substantially uniformly suspended within the carrier oil.

2. The CBD mineral salve of claim 1, wherein the hemp oil is present in about 5% to about 20% by volume of the CBD mineral salve.

3. The CBD mineral salve of claim 1, wherein the magnesium oil is present in about 5% to about 40% by volume of the CBD mineral salve.

4. The CBD mineral salve of claim 1, wherein the carrier oil is present in about 60% to about 80% by volume of the CBD mineral salve.

5. The CBD mineral salve of claim 4, wherein the carrier oil is selected from the group consisting of: bee wax, carauba wax, Olive oil, Hempseed oil, Coconut oil, Avocado oil, Coco Butter, Mango Butter, Shea Butter (raw yellow, refined white), Jojoba, and any combination thereof.

6. The CBD mineral salve of claim 5, wherein the carrier oil comprises bee wax or carauba wax, and at least one additional carrier oil selected from the group consisting of: Olive oil, Hempseed oil, Coconut oil, Avocado oil, Coco Butter, Mango Butter, Shea Butter (raw yellow, refined white), Jojoba, and any combination thereof.

7. The CBD mineral salve of claim 1, wherein the black pepper oil is present in about 0.5% to about 3% by volume of the CBD mineral salve.

8. The CBD mineral salve of claim 1, wherein the black pepper oil is present in about 0.5% to about 2% by volume of the CBD mineral salve.

9. The CBD mineral salve of claim 1, wherein the turmeric oil or curcumin oil is present in about 0.5% to about 3% by volume of the CBD mineral salve.

10. The CBD mineral salve of claim 1, wherein the turmeric oil or curcumin oil is present in about 1% to about 3% by volume of the CBD mineral salve.

11. The CBD mineral salve of claim 1, wherein the one or more essential oil is present in about 2% to about 10% by volume of the CBD mineral salve.

12. The CBD mineral salve of claim 1, wherein the one or more essential oil is selected from the group consisting of: eucalyptus, peppermint, citrus, lemongrass, lavender, and any combination thereof.

13. The CBD mineral salve of claim 1, further comprising a natural preservative.

14. The CBD mineral salve of claim 13, wherein the natural preservative is selected from the group consisting of: Populus tremuloides bark extract; willow bark extract; leuconostoc ferment filtrate; Phytocide Elderberry; a combination of gluconolactone and sodium benzoate; a combination of benzyl alcohol, salicylic acid, glycerin, and sorbic acid; radish root; lactobacillus ferment; and any combination thereof.

15. A method of reducing or treating arthritic pain or muscle pain, inflammation, and/or a skin condition by topically administering the CBD mineral salve of claim 1.

16. The method of claim 15, wherein the skin condition is an inflammatory skin condition.

17. The method of claim 15, wherein the CBD mineral salve is administered topically one to six times daily.

18. A method of treating and/or healing a burn on skin of a subject by topically administering the CBD mineral salve of claim 1 to the burn.

19. A method of making the CBD mineral salve of claim 1, the method comprising the steps of:
i) supplying raw hemp;
ii) processing the raw hemp to make hemp oil;
iii) supplying magnesium chloride flakes;
iv) processing the magnesium chloride flakes with water to make magnesium oil;
v) melting the at least one carrier oil to a liquid state;
vi) mixing the hemp oil, the melted carrier oil, black pepper oil, turmeric oil or curcumin oil, the natural preservative, and the one or more essential oils in a blending unit; and
v) blending the mixture for an amount of time at about 120° C. to about 170° C. to provide a substantially uniformly blended CBD mineral salve.

20. A method of making the CBD mineral salve of claim 19, wherein the magnesium oil is substantially uniformly suspended in the carrier oil.
